(19) United States
(12) Patent Application Publication
(10) Pub. No.: US 2003/0163001 A1
(43) Pub. Date: Aug. 28, 2003

(54) Method for oximating organic carbonyl compounds and/or ch-acid compounds

(76) Inventors: Hanns Wurziger, Darmstadt (DE); Joeran Stoldt, Darmstadt (DE); Guido Pieper, Mannheim (DE); Norbert Schwesinger, Eching (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/239,568

(22) PCT Filed: Mar. 6, 2001
(86) PCT No.: PCT/EP01/02513

(30) Foreign Application Priority Data
Mar. 30, 2000 (DE) 100 15 518.9

(51) Int. Cl. C07C 249/08

(52) U.S. Cl. 564/259

(57) ABSTRACT

The invention relates to a method for oximating organic carbonyl compounds and/or CH-acid compounds, wherein at least one organic carbonyl compound and/or CH-acid compound in a liquid or dissolved form is mixed with at least one oximating agent in a liquid or dissolved form in at least one microreactor, is reacted during a residence time and the organic oxime thus formed is optionally isolated from the reaction mixture.

The present invention relates to a method for oximating organic carbonyl compounds and/or CH-acidic compounds.

The oximation of organic carbonyl compounds and/or CH-acidic compounds is a method which is carried out very frequently in the chemical industry and whose great importance is also reflected in numerous publications on this subject.

However, the performance of oximations of organic carbonyl compounds and/or CH-acidic compounds on an industrial scale is associated with safety problems and risks. Firstly, use is frequently made of relatively large amounts of highly toxic chemical substances, which even alone represent a considerable risk to humans and the environment, and secondly oximations frequently proceed very highly exothermically, which means that there is an increased risk of explosion when these reactions are carried out on an industrial scale. The attainment of official approval in accordance with the German Federal Emissions Protection Act (BimschG) for the operation of plants for the oximation of organic carbonyl compounds and/or CH-acidic compounds on an industrial scale is therefore associated with considerable effort.

The object of the present invention is therefore to provide a method for oximating organic carbonyl compounds and/or CH-acidic compounds which avoids the above-mentioned disadvantages. The aim is, in particular, for it to be possible for this method to be carried out in a simple, reproducible manner with increased safety for humans and the environment and with good yields and for the reaction conditions to be readily controllable. This object is achieved, surprisingly, by the method according to the invention for oximating organic carbonyl compounds and/or CH-acidic compounds, in which at least one organic carbonyl compound and/or CH-acidic compound in liquid or dissolved form is mixed with at least one oximating agent in liquid or dissolved form in at least one microreactor and reacted for a residence time, and the organic oxime formed is, if desired, isolated from the reaction mixture.

Advantageous embodiments of the method according to the invention are described in the sub-claims.

In accordance with the invention, individual organic carbonyl compounds, CH-acidic compounds or mixtures of these compounds are oximated by the claimed method. In a preferred embodiment, in each case only one organic carbonyl compound or CH-acidic compound is employed in the method according to the invention.

For the purposes of the invention, a microreactor is a reactor having a volume of ≦1000 μl in which the liquids and/or solutions are intimately mixed at least once. The volume of the reactor is preferably ≦100 μl, particularly preferably ≦50 μl.

The microreactor is preferably made from thin silicon structures connected to one another.

The microreactor is preferably a miniaturised flow reactor, particularly preferably a static micromixer. The microreactor is very particularly preferably a static micromixer as described in the interational patent application with the publication number WO 96/30113, which is incorporated herein by way of reference and is regarded as part of the disclosure.

A microreactor of this type has small channels in which liquids and/or chemical compounds in the form of solutions are mixed with one another by means of the kinetic energy of the flowing liquids and/or solutions.

The channels of the microreactor preferably have a diameter of from 10 to 1000 μm, particularly preferably from 20 to 800 μm and very particularly preferably from 30 to 400 μm.

The liquids and/or solutions are preferably pumped into the microreactor in such a way that they flow through the latter at a flow rate of from 0.01 μl/min to 100 ml/min, particularly preferably from 1 μl/min to 1 ml/min.

In accordance with the invention, the microreactor is preferably heatable.

In accordance with the invention, the microreactor is preferably connected via an outlet to at least one residence zone, preferably a capillary, particularly preferably a heatable capillary. After mixing in the microreactor, the liquids and/or solutions are fed into this residence zone or capillary in order to extend their residence time.

For the purposes of the invention, the residence time is the time between mixing of the starting materials and work-up of the resultant reaction solution for analysis or isolation of the desired product(s).

The residence time necessary in the method according to the invention depends on various parameters, such as, for example, the temperature or reactivity of the starting materials. It is possible for the person skilled in the art to match the residence time to these various parameters and thus to achieve an optimum course of the reaction.

The residence time of the reaction solution in the system used, comprising at least one microreactor and, if desired, a residence zone can also be set through the choice of the flow rate of the liquids and/or solutions employed.

The reaction mixture is likewise preferably passed through two or more microreactors connected in series. This achieves an extension of the residence time, even at an increased flow rate, and the oximation reaction components employed are reacted to such an extent that an optimum product yield of the desired organic oxime(s) is achieved.

In a further preferred embodiment, the reaction mixture is passed through two or more microreactors arranged in parallel in order to increase the throughput.

In another preferred embodiment of the method according to the invention, the number and arrangement of the channels in one or more microreactors are varied in such a way that the residence time is extended, likewise resulting in an optimum yield of the desired organic oxime(s) at an increased flow rate.

The residence time of the reaction solution in the microreactor, where appropriate in the microreactor and the residence zone, is preferably ≦15 hours, particularly preferably ≦3 hours and very particularly preferably ≦1 hour.

The method according to the invention can be carried out in a very broad temperature range, which is essentially restricted by the heat resistance of the materials employed for the construction of the microreactor, any residence zone and further constituents, such as, for example, connections and seals, and by the physical properties of the solutions and/or liquids employed. The method according to the invention is preferably carried out at a temperature of from −100 to +2500° C., particularly preferably from −78 to +150° C. and very particularly preferably from 0 to +40° C.

The method according to the invention can be carried out either continuously or batchwise. It is preferably carried out continuously.

For carrying out the method according to the invention for oximating organic carbonyl compounds and/or CH-acidic compounds, it is necessary for the oximation reaction to be carried out as far as possible in the homogeneous liquid phase containing no or only very small solid particles, since otherwise the channels present in the microreactors become blocked.

The course of the oximation reaction in the method according to the invention can be followed using various analytical methods known to the person skilled in the art and if necessary regulated. The course of the reaction is preferably followed by chromatography, particularly preferably by high-pressure liquid chromatography, and if necessary regulated. Control of the reaction is significantly improved in the method according to the invention compared with known methods.

After the reaction, the organic oximes formed are isolated if desired. The product(s), formed is (are) preferably isolated from the reaction mixture by extraction and/or precipitation.

The organic carbonyl compounds or CH-acidic compounds employed can be any organic compounds from the above-mentioned classes of substance which are known to the person skilled in the art and are suitable as substrate for oximation reactions. The organic carbonyl compounds or CH-acidic compounds are preferably selected from aliphatic, aromatic or heteroaromatic aldehydes, ketones or CH-acidic compounds.

The aliphatic aldehyde, ketone or CH-acidic compounds employed can be any aliphatic compounds from the above-mentioned classes of substance which are known to the person skilled in the art and are suitable as substrate for oximation reactions. This also includes straight-chain, branched, cyclic, saturated and unsaturated compounds.

The aromatic aldehyde, ketone or CH-acidic compounds employed can be any aromatic compounds from the above-mentioned classes of substance which are known to the person skilled in the art and are suitable as substrate for oximation reactions. For the purposes of the invention, this thus includes compounds and/or derivatives which have a monocyclic and/or polycyclic homoaromatic basic structure or a corresponding moiety, for example in the form of substituents.

The heteroaromatic aldehyde, ketone or CH-acidic compounds employed can be any heteroaromatic compounds from the above-mentioned classes of substance which are known to the person skilled in the art and are suitable as substrate for oximation reactions and contain at least one heteroatom. For the purposes of the invention, heteroaromatic compounds include heteroaromatic compounds and/or derivatives thereof which have at least one monocyclic and/or polycyclic heteroaromatic basic structure or a corresponding moiety, for example in the form of substituents. Heteroaromatic basic structures or moieties preferably include at least one oxygen, nitrogen and/or sulfur atom.

Oximating agents which can be employed in the method according to the invention are all oximating agents which are known to the person skilled in the art and are suitable for the oximation of organic carbonyl compounds and/or CH-acidic compounds, or a mixture of at least two components. Preferably, only one oximating agent is employed in each case.

In a further preferred embodiment, at least one oximating agent selected from hydroxylamine, hydroxylamine O-ethers, salts of nitrous acid, organic nitrites or a mixture of at least two of these oximating agents is employed.

Examples of preferred organic nitrites include tert-butyl nitrite, n-pentyl nitrite, isopentyl nitrite, isopropyl nitrite or a mixture of at least two of these nitrites.

The molar ratio between the organic carbonyl compound and/or CH-acidic compound employed and the oximating agent employed depends in the method according to the invention on the reactivity of the organic carbonyl compounds, CH-acidic compounds and oximating agents employed. The molar ratio between the oximating agent and the organic carbonyl compound and/or CH-acidic compound is preferably equimolar. In a further preferred embodiment, the oximating agent is used in a 1.2-fold to 2-fold molar excess, particularly preferably in a 1.3-fold to 1.9-fold excess, very particularly preferably in a 1.4-fold to 1.8-fold excess, based on the organic carbonyl compound and/or the CH-acidic compound.

The selectivity of the reaction itself depends on a number of further parameters in addition to the concentration of the reagents employed, such as, for example, the temperature, the type of oximating agent used or the residence time. It is possible for the person skilled in the art to match the various parameters to the respective oximation reaction in such a way that the desired oximated product(s) is (are) obtained.

It is essential for the method according to the invention that the organic carbonyl compounds, CH-acidic compounds and oximating agents employed are either themselves liquid or are in dissolved form. If these compounds are not already themselves in liquid form, they therefore have to be dissolved in a suitable solvent before the method according to the invention is carried out. The solvents employed are preferably water, ethers, particularly preferably diethyl ether, methyl tert-butyl ether, tetrahydrofuran or dioxane, aromatic solvents, particularly preferably toluene, xylenes, ligroin or phenyl ether, halogenated solvents, particularly preferably dichloromethane, chloroform, 1, 2-dichloroethane or 1, 1, 2, 2-tetrachloroethane, or mixtures thereof.

It is also possible to oxidise the oximes prepared by the method according to the invention in at least one microreactor to give nitrile oxides, which react further in situ with suitable dipolarophiles or dipolarophilic groups in intra-or intermolecular 1, 3-dipolar cycloadditions with formation of heterocyclic compounds.

The dipolarophiles reacted can be any dipolarophiles known to the person skilled in the art which are suitable for 1,3-dipolar cycloadditions.

The present invention also includes the conversion of all organic carbonyl compounds and/or CH-acidic compounds known to the person skilled in the art into nitrile oxides containing at least one dipolarophilic functional group which is able to react in 1,3-dipolar cycloadditions. It is possible here for the organic compound in question to contain only one dipolarophilic group or 1, 3-dipolar group or a combination of at least two dipolarophilic or dipolarophilic groups, which may in each case be identical or different. Preferably, only one 1, 3-dipolar group and only one dipolarophilic functional group is present.

These groups can react in intramolecular 1, 3-dipolar cycloadditions or, if the intramolecular reaction is not possible, for example for steric reasons, in intermolecular cycloadditions with formation of heterocyclic compounds.

Various heterocyclic compounds formed by this route can be employed in the synthesis of organic compounds which are very highly suitable as starting materials for the search for novel pharmaceutical active ingredients by combinatorial chemistry.

The oxidants for conversion of the oximes into nitrile oxides include all compounds known to the person skilled in the art which can be used as oxidants in the above-mentioned reaction. The oxidants can be employed either in pure form or in the form of mixtures. Preferably, in each case only one oxidant is used in the method according to the invention.

In a further preferred embodiment, the oxime oxidants employed are N-halogenated succinimides, particularly preferably succinimides which are substituted by Cl, Br or I atoms, or free halogens, particularly preferably Cl₂, Br₂ or I₂, or salts of hypohalous acids, particularly preferably sodium hypochlorite, or a mixture of the above-mentioned compounds.

Furthermore, the microreactor(s) in which the oximation reaction has been carried out can be connected directly to at least one microreactor in which the oxidation of the resultant oxime to the corresponding nitrile oxide and the 1, 3-dipolar cycloaddition with formation of the heterocyclic compounds takes place without the oxime formed as an intermediate being isolated.

In the method according to the invention, the risk to humans and the environment caused by escaping chemicals is considerably reduced, thus increasing safety during handling of hazardous materials. The oximation of aliphatic, aromatic or heteroaromatic organic carbonyl compounds and/or CH-acidic compounds by the method according to the invention furthermore enables better control of the reaction conditions, such as, for example, reaction duration and reaction temperature, than is possible in the conventional methods. Furthermore, the risk of explosions in very highly exothermic oximation reactions is significantly reduced on use of the method according to the invention. The temperature can be selected and kept constant in each individual volume unit of the system. The course of the oximation reactions can be regulated very rapidly and precisely in the method according to the invention. The resultant oximated products can thus be obtained in very good and reproducible yields.

It is also particularly advantageous that the method according to the invention can be carried out continuously. This makes the method faster and less expensive than conventional methods, and it is possible to prepare any desired amounts of the oximated organic compounds without major measurement and regulation complexity. The oximes can be converted into heterocyclic compounds very effectively and with high purity in at least one further micromixer, either after isolation of the oxime formed in the first reaction step or by direct reaction of this oxime without interim isolation. Various of these heterocyclic compounds are important precursors for the synthesis of compounds which are very highly suitable for the search for novel active ingredients by combinatorial chemistry.

The invention is explained below with reference to examples. These examples serve merely to explain the invention and do not restrict the general inventive idea.

### EXAMPLES

### Example 1

Oximation of 5-bromo-2-allyloxybenzaldehyde to 5-bromo-2-allyloxybenzaldoxime

The oximation of 5-bromo-2-allyloxybenzaldehyde by means of hydroxylamine hydrochloride was carried out in a static micromixer (Technical University of Ilmenau, Faculty of Machine Construction, Dr. Norbert Schwesinger, Postfach 100565, D-98684 Ilmenau) having a physical size of 40 mm×25 mm×1 mm which had a total of 11 mixing stages each with a volume of 0.125 μl. The total pressure loss was about 1000 Pa.

The static micromixer was connected via an outlet and an Omnifit medium-pressure HPLC connector (Omnifit, Great Britain) to a Teflon capillary having an internal diameter of 0.49 mm and a length of 0.5 m. The reaction was carried out at room temperature or at 10° C. In the latter case, the temperature of the static micromixer and the Teflon capillary was controlled in an ethanol-filled double-wall vessel thermostatted to 10° C.

A 2 ml disposable syringe was filled with part of a solution of 0.8 g (12 mmol) of hydroxylamine hydrochloride and 75 ml of 1 N sodium hydroxide solution, and a further 2 ml syringe was filled with part of a solution of 2.2 g (9 mmol) of 5-bromo-2-allyloxybenzaldehyde in 75 ml of dioxane. The contents of the two syringes were subsequently transferred into the static micromixer by means of a metering pump (Harvard Apparatus Inc., Pump 22, South Natick, Mass., U.S.A.). Before the reaction was carried out, the experimental arrangement was calibrated with respect to the dependence of the residence time on the pump flow rate. The pump rate was set in such a way that a residence time of 5, 10 or 20 minutes was reached. The reactions were monitored with the aid of a Merck Hitachi LaChrom HPLC instrument. The starting material:product ratio obtained through this reaction was likewise determined with the aid of HPLC on the above instrument.

### Example 2

Oxidation of 5-bromo-2-allyloxybenzaldoxime to 5-bromo-2-allyloxybenzonitrile oxide and 1,3-dipolar cycloaddition to 8-bromo-3α,4-dihydro-3H-[1]-benzopyrano[4, 3-c]isoxazole

The 5-bromo-2-allyloxybenzaldoxime prepared in Example 1 was oxidised using a sodium hypochlorite solution in a microreactor having the characteristics indicated in Example 1 at each of room temperature, 10° C. and 0° C. and reacted in an intramolecular 1,3-dipolar cycloaddition. In the case of the two latter temperatures, the temperature of the static micromixer and the Teflon capillary was controlled in an ethanol-filled double-wall vessel thermostatted to 10° C. or 0° C.

A 2 ml disposable syringe was filled with part of a solution of 0.5 g (2 mmol) of 5-bromo-2-allyloxybenzaldoxime and 10 ml of dichloromethane, and a further 2 ml syringe was filled with an approximately 10% aqueous sodium hypochlorite solution. The contents of the two syringes were subsequently transferred into the static micromixer by means of a metering pump (Harvard Apparatus Inc., Pump 22, South Natick, Mass., U.S.A.). Determination of the yield as a function of the residence time was not possible in the case of the present reaction since the oxidation of the aldoxime to the corresponding nitrile oxide proceeded extremely quickly. The yield and purity of the desired product were determined by HPLC on a Merck Hitachi LaChrom HPLC instrument.

### Example 3

Combined oximation of 5-bromo-2-allyloxybenzaldehyde to 5-bromo-2-allyloxybenzaldoxime, oxidation to 5-bromo-2-allyloxybenzonitrile oxide and 1, 3-dipolar cycloaddition to 8-bromo-3α,4-dihydro-3H-[1]-benzopyrano [4, 3c]-2-isoxazoline

The oximation was carried out at room temperature as indicated in Example 1. The pump rate was set in such a way that a residence time of 25 minutes resulted. The outlet capillary connected to the reactor was connected to the first inlet of a second static micromixer. The second inlet was connected to a syringe, which was filled with sodium hypochlorite solution as described in Example 2. This solution was transferred into the second reactor by means of a further pump of the above-mentioned type. The pump rate thereof was set to a value which was a factor of two higher than the pump rate of the first pump. The technical data for the two reactors connected in series were as in Example 1.

The yield and purity of the desired product were determined by HPLC on a Merck Hitachi LaChrom HPLC instrument.

1. Method for oximating organic carbonyl compounds and/or CH-acidic compounds, characterised in that at least one organic carbonyl compound and/or at least one CH-acidic compound in liquid or dissolved form is mixed with at least one oximating agent in liquid or dissolved form in at least one microreactor and reacted for a residence time, and the organic oxime formed is, if desired, isolated from the reaction mixture.

2. Method according to claim 1, characterised in that the microreactor is a miniaturised flow reactor.

3. Method according to claim 1 or 2, characterised in that the microreactor is a static micromixer.

4. Method according to one of claims 1 to 3, characterised in that the microreactor is connected via an outlet to a capillary, preferably a heatable capillary.

5. Method according to one of claims 1 to 4, characterised in that the volume of the microreactor is ≦100 μl, preferably ≦50 μl.

6. Method according to one of claims 1 to 5, characterised in that the microreactor is heatable.

7. Method according to one of claims 1 to 6, characterised in that the microreactor has channels having a diameter of from 10 to 1000 μm, preferably from 20 to 800 μm, particularly preferably from 30 to 400 μm.

8. Method according to one of claims 1 to 7, characterised in that the reaction mixture flows through the microreactor at a flow rate of from 0.01 μl/min to 100 ml/min, preferably from 1 μl/min to 1 ml/min.

9. Method according to one of claims 1 to 8, characterised in that the residence time of the compounds employed in the microreactor, where appropriate in the microreactor and the capillaries, is ≦15 hours, preferably ≦3 hours, particularly preferably ≦1 hour.

10. Method according to one of claims 1 to 9, characterised in that it is carried out at a temperature of from −100 to +250° C., preferably from −78 to +150° C., particularly preferably from 0 to +40° C.

11. Method according to one of claims 1 to 10, characterised in that the course of the reaction is followed by chromatography, preferably by high-pressure liquid chromatography, and if necessary regulated.

12. Method according to one of claims 1 to 11, characterised in that the organic carbonyl compounds are selected from aliphatic, aromatic or heteroaromatic aldehydes or ketones.

13. Method according to one of claims 1 to 12, characterised in that the CH-acidic compounds are selected from aliphatic, aromatic or heteroaromatic compounds.

14. Method according to one of claims 1 to 13, characterised in that the oximating agent used is at least one compound selected from hydroxylamine, hydroxylamine O-ethers, salts of nitrous acid, organic nitrites or a mixture of these oximating agents.

15. Method according to claim 14, characterised in that the organic nitrite used is tert-butyl nitrite, n-pentyl nitrite, isopentyl nitrite, isopropyl nitrite or a mixture thereof.

16. Method according to one of claims 1 to 14, characterised in that the molar ratio between the oximating agent and the organic carbonyl compound and/or CH-acidic compound is equimolar, or in that the oximating agent is employed in a 1.2-fold to 2-fold molar excess, particularly preferably in a 1.3-fold to 1.9-fold excess, very particularly preferably in a 1.4-fold to 1.8-fold excess, based on the organic carbonyl compound and/or the CH-acidic compound.
